(19) United States
(12) Patent Application Publication
(10) Pub. No.: US 2015/0190542 A1
(43) Pub. Date: Jul. 9, 2015

(54) FRAGRANCE DIFFUSION DEVICE WITH A MULTITUDE OF OPENINGS

(71) Applicant: TECHNIPLAST, LOUVIERS (FR)

(72) Inventors: Jean-Philippe LAMBOUX, Saint Didier des Bois (FR); Frederic LEQUERE, Nogent le Sec (FR)

(21) Appl. No.: 14/591,270

(22) Filed: Jan. 7, 2015

(30) Foreign Application Priority Data
Jan. 7, 2014 (FR) 14 50072

(51) Int. Cl. A61L 9/12

(52) U.S. Cl. CPC A61L 9/12

(57) ABSTRACT

A fragrance diffusion device includes:
two opposing walls between them defining a cavity which extends transversally as far as a peripheral wall provided with an opening for diffusing fragrance to outside the device,
several openings formed in each wall and which pass through same so as to allow, in the case of a first wall, the fragrance to pass into the cavity from a first zone which is adjacent to the first wall and separated from the cavity by this wall and, in the case of the second wall, the fragrance to pass from the cavity to a second zone which is adjacent to the second wall and separated from the cavity by this second wall,
a duct passing through each of the two walls and extending into the zone adjacent to the relevant wall so as to place each zone in communication with the cavity.

The invention relates to a device for diffusing a fragrance such as a scent.

Document FR 2 958 854 discloses a fragrance diffuser comprising:
two pouring plugs mounted top to tail and each having a flow tube,
an insert of porous material fitted between the two pouring plugs,
an external ring provided, facing the insert, with at least one fragrance diffusion orifice.

Each pouring plug also comprises a flow hole which allows the fragrance to pass into the porous insert and then to the outside of the device. The fragrance is, for example, contained in a bottle arranged above the diffuser.

Although satisfactory, this diffuser could nevertheless be simplified.

To that end, the invention is aimed at a fragrance diffusion device, characterized in that it comprises:
two opposing walls spaced apart from one another along a vertical longitudinal axis L so as to define between them a cavity C which extends transversally as far as a peripheral wall provided with at least one opening for diffusing fragrance to outside the device, said at least one opening placing the cavity in communication with the outside of the device,
several openings formed in each of the two opposing walls and which pass through the relevant wall in such a way as to allow, in the case of a first of the two walls which is situated above the second wall, the passage of the liquid fragrance from a first zone Z1 into the cavity so that said fragrance can be diffused to outside the device via said at least one diffusion opening, the first zone being adjacent to said first wall, above the latter and separated from the cavity by this first wall and, in the case of the second of the two walls, the passage of the liquid fragrance from the cavity to a second zone Z2 which is adjacent to said second wall, underneath the latter and separated from the cavity by this second wall,
at least one duct which passes through each of the two opposing walls and extends into the zone adjacent to the relevant wall so as to place each zone adjacent to each wall in communication with the cavity, said at least one duct formed in the first wall being an air intake duct which directly takes in air from the outside of the device via said at least one opening and the cavity, whereas said at least one duct formed in the second wall is a flow duct for the liquid fragrance coming from the cavity.

In the device according to one embodiment of the invention, the fragrance (e.g. scent) passes through the openings in the first wall (the upper wall when the device is in a vertical operating position, the liquid fragrance being contained in the first zone, notably an inverted upper bottle), and when the cavity is free of any obstacle, drops into the cavity (diffusion space), producing a drop by drop dripping effect. The drops fall under gravity onto the opposing second wall (lower wall is referred to as collector) and flow through said at least one duct connected to this wall into the second zone adjacent thereto (notably into a lower bottle).

This configuration of the device thus provides, using the same means, both diffusion of fragrance into the cavity and to the outside and collection of the drops in order to produce the drop by drop dripping under the device (notably into a lower bottle). The device according to the invention is therefore simpler than that of the prior art in which the diffusion and dripping functions are separate. No porous materials that have to be installed in the cavity are provided either, making the device simpler and less expensive.

It will be noted that the device according to one embodiment of the invention may comprise a cavity that may or may not be free of any obstacle between the two opposing walls.

Fragrance is diffused into the cavity and then to the outside of the device and liquid fragrance not diffused in the cavity passes from the cavity to said at least one flow duct. The liquid fragrance therefore does not pass directly from the first zone to the second zone as it does in the prior art.

Moreover, in the abovementioned prior art, in order to achieve the drop by drop dripping effect, very small holes (e.g.: 0.2 to 0.5 mm) are needed and these have a tendency to become blocked in the presence of fatty products included in the composition of fragrances (scents). With the device according to the invention, the multitude of openings in the opposing walls limits this phenomenon.

Unlike the device of the abovementioned prior art, the intake air that compensates for the volume of fragrance flowing through the openings in the first wall comes from outside the device and passes through said at least one fragrance diffusing opening then into the cavity and into said at least one air intake duct. This means that the compensation for air in the bottle that is dispensing its fragrance can be dissociated from the flow of this fragrance and therefore from the drop by drop dripping. In the prior art, the bottles communicate with one another via air-passage and flow tubes. Thus, the pressure or depression in one or other of the bottles has an influence on the drop by drop dripping function (for example when pressure equilibrium needs to be established), something which is not the case with the device according to the invention.

It will be noted that said at least one duct passing through each wall projects exclusively into the relevant adjacent zone and not into the cavity between the walls so that it does not impede the flow of the liquid in the flow duct or flow ducts of the second wall.

According to other possible features considered in isolation or in combination with one another:
said at least one air intake duct extends in the first zone Z1 over a height suited to the open end of said at least one air intake duct being at a position above the level of liquid fragrance contained in the first zone Z1; this arrangement guarantees the function of taking in air from outside the device;
the openings of one of the two opposing walls are geometrically offset in relation to the openings of the other wall; the fact that the openings in a wall do not face the openings in the opposing wall makes it possible to prevent drops emanating from the upper openings from falling onto the lower openings, as they would risk blocking these openings;
at least certain openings formed in the two opposing walls are each extended by a neck which projects into the cavity with respect to the relevant wall; this arrangement makes it possible to prevent drops from obstructing these openings in the second wall when they drop onto this wall and flow over it; these openings in the second wall allow air present in the lower bottle to be removed while non-diffused liquid fragrance flows into said bottle;
the device comprises several liquid-fragrance flow-retarding elements each comprising a body of elongate overall shape, the bodies or obstacles extending into the cavity so as each to connect two respective openings of the two opposing walls, thus allowing the liquid fragrance to be guided via each flow-retarding element from an opening in the first wall to an opening in the second wall; these bodies or obstacles retard the flow by diverting it from its direct, notably vertical, path from the first to the second wall; these bodies or obstacles for example offer several simultaneous possible paths for the flow rather than just one (direct path);
the bodies of the fragrance flow-retarding elements are formed of several tubes which extend into the cavity in such a way that each tube connects together two respective openings of the two opposing walls, thus allowing the liquid fragrance to be guided via each tube from an opening in the first wall to an opening in the second wall; in this configuration, the tubes retard the flow of the drops of fragrance, thus prolonging the time they spend in the cavity and therefore the diffusion phenomenon;
each tube comprises, arranged along its length, at least two internal compartments which are separated from one another by a transverse partition, said tube being pierced along its length by a plurality of lateral orifices which allow the liquid fragrance present in one compartment arranged near a first wall to leave same, flow along the external face of said tube until reaching the second wall and then said at least one duct formed in said second wall. This arrangement creates as it were chicanes for the flow of liquid fragrance, preventing direct passage vertically from one opening to the other; the time for which the fragrance is present in the cavity (notably though stagnation and flowing) is thus increased, making it possible to prolong the diffusion effect and therefore improve same;
the fragrance flow-retarding elements extend into the cavity along the longitudinal axis;
the device comprises, on either side of the two walls, in each zone adjacent to each wall, fixing means which are each intended to fix the neck of a bottle to the device;
each wall has a concave overall shape with the concave side facing towards the cavity and comprises a central region;
said at least one duct is situated in the central region;
the fragrance flow-retarding elements are arranged around said at least one duct;
the device comprises one or more elements made of absorbent material, which are formed on each of the two opposing walls; this or these elements are able to absorb the liquid which drops onto them from the upper bottle in order temporarily to hold the liquid there, thus preventing the risks of running when the device is inverted.

According to a second aspect, the invention is also aimed at a fragrance diffusion system, characterized in that it comprises:
a diffusion device as briefly explained hereinabove,
an upper first bottle having a first neck the opening of which is positioned facing the first of the two opposing walls referred to as the upper wall; a lower second bottle having a second neck the opening of which is positioned facing the second wall referred to as the lower wall, the upper first bottle comprising the liquid fragrance which, by flowing drop by drop through the openings on the one hand causes the fragrance to diffuse into the cavity and to outside of the device via the said at least one opening, and on the other hand causes dripping drop by drop into the lower second bottle.

This system has the same advantages and features as those mentioned in relation to the diffusion device mentioned above and these will therefore not be repeated here.

According to a third aspect, the invention is aimed at a method for diffusing a fragrance in a fragrance diffusion device, characterized in that the device comprises:
two opposing walls spaced apart from one another along a vertical longitudinal axis L so as to define between them a cavity C which extends transversally as far as a peripheral wall provided with at least one opening for diffusing fragrance to outside the device, said at least one opening placing the cavity in communication with the outside of the device,
several openings formed in each of the two opposing walls and which pass through the relevant wall in such a way as to allow, in the case of a first of the two walls which is situated above the second wall, the passage of the liquid fragrance from a first zone Z1 into the cavity so that said fragrance can be diffused to outside the device via said at least one diffusion opening, the first zone being adjacent to said first wall, above the latter and separated from the cavity by this first wall and, in the case of the second of the two walls, the passage of the liquid fragrance from the cavity to a second zone Z2 which is adjacent to said second wall, underneath the latter and separated from the cavity by this second wall,
at least one duct which passes through each of the two opposing walls and extends into the zone adjacent to the relevant wall so as to place each zone adjacent to each wall in communication with the cavity, said at least one duct formed in the first wall being an air intake duct whereas said at least one duct formed in the second wall is a flow duct for the fragrance coming from the cavity, according to the method, the liquid fragrance contained in the first zone Z1 flows through the openings in the first wall so that, on the one hand, the fragrance diffuses into the cavity C and to outside the device via said at least one opening and, on the other hand, the remaining liquid fragrance that has not diffused flows into the second zone Z2 via said at least one duct, the flow of liquid fragrance through the openings in the first wall being permitted as a result of the intake of external air via said at least one opening, the cavity C and said at least one air intake duct.

According to other possible features of the method:
the flow of liquid fragrance from the first zone through the openings in the first wall is diverted, as it passes into the cavity, from a direct path in order to reach the second wall so as to increase the time for which said flow is present in said cavity; diverting the flow has a retarding effect on the flow; increasing the time present in the cavity has the effect of prolonging, and therefore of improving, the effect of diffusing the fragrance to outside the device;
the flow of liquid fragrance is diverted by encountering a number of obstacles present in the cavity C; the flow thus runs alongside or passes through the obstacles or bodies extending into the cavity and which divert said flow from what in the absence of such obstacles or bodies would be a more direct path;
the diffusion of fragrance takes place in a fragrance diffusion system comprising:
a fragrance diffusion device,
an upper first bottle having a first neck the opening of which is positioned facing the first of the two opposing walls referred to as the upper wall; a lower second bottle having a second neck the opening of which is positioned facing the second wall referred to as the lower wall, the upper first bottle comprising the liquid fragrance. The fragrance, by flowing drop by drop through the openings causes, on the one hand, the fragrance to diffuse into the cavity C and to outside the device via said at least one opening and, on the other hand, to drip drop by drop into the lower second bottle.

Other features and advantages will become apparent during the course of the following description, given solely by way of nonlimiting example and made with reference to the attached drawings in which:

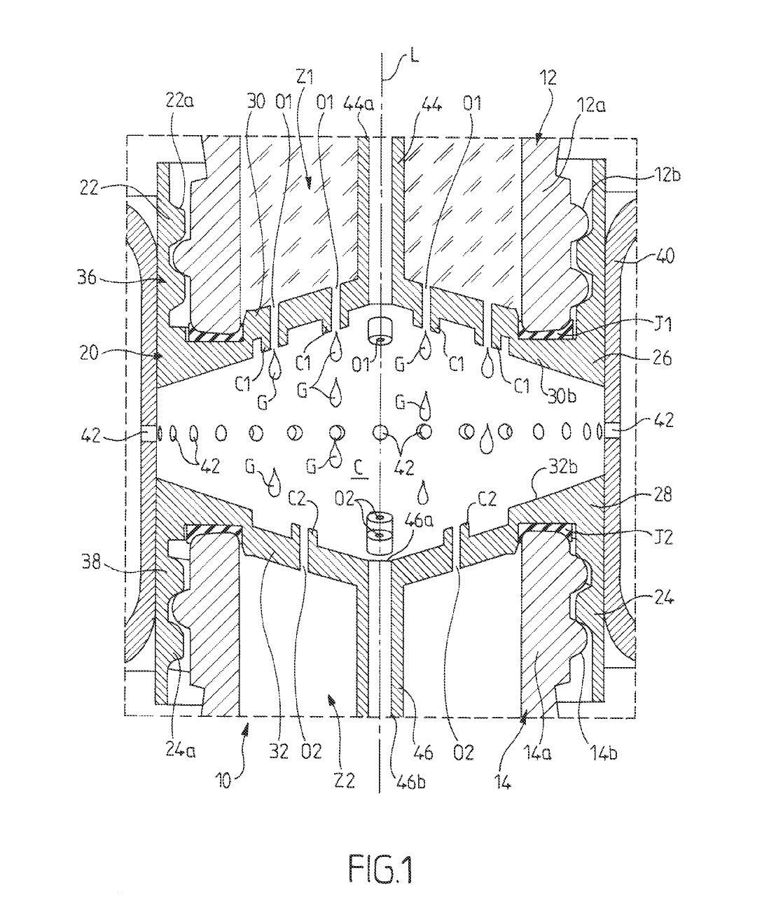

FIG. 1 is a schematic overall view in axial section of a fragrance diffusion device according to a first embodiment of the invention;

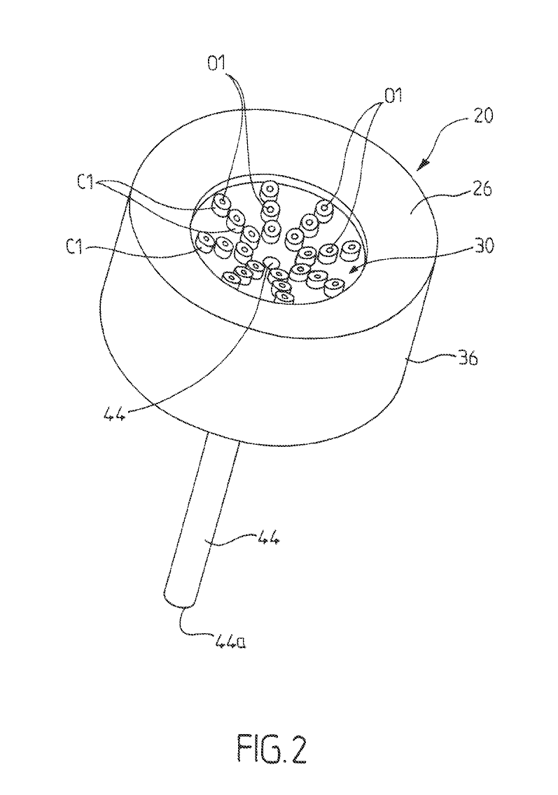

FIG. 2 is a view from beneath of the device of FIG. 1 inverted with a different distribution of openings in the wall 30;

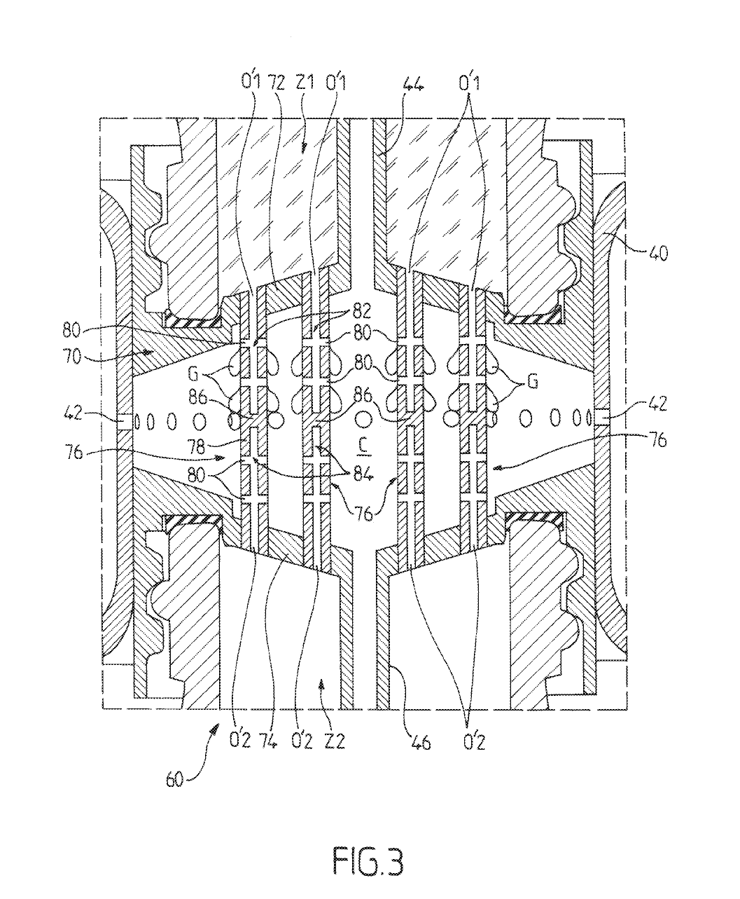

FIG. 3 is a schematic overall view in axial section of a fragrance diffusion device according to a second embodiment of the invention;

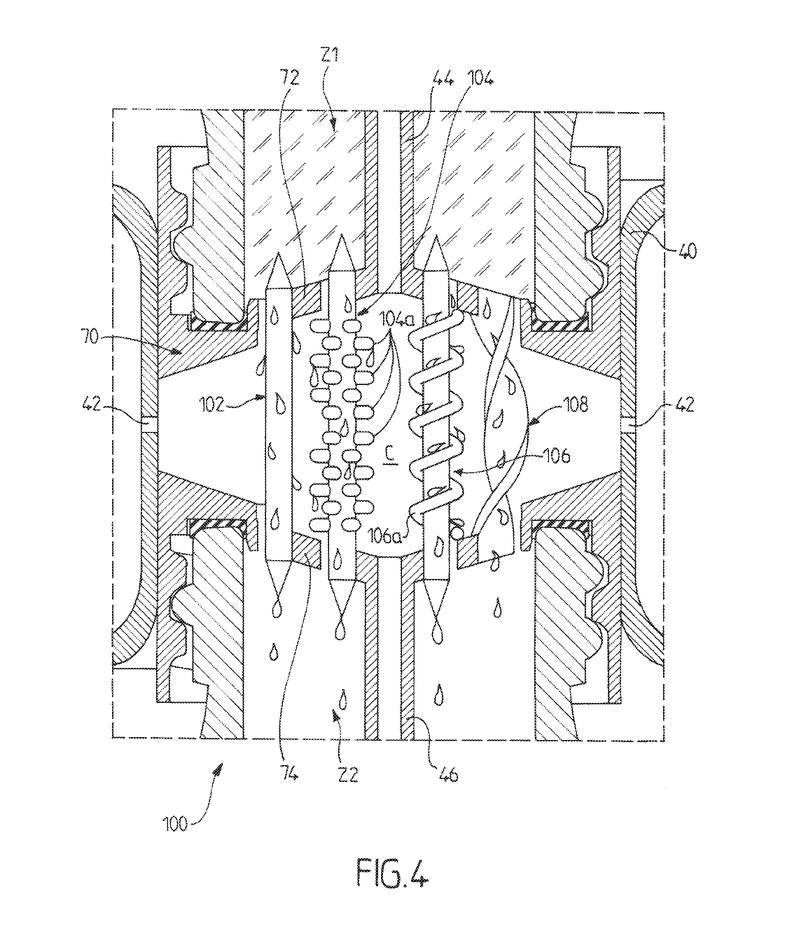

FIG. 4 is a schematic view in axial section of a fragrance diffusion device which artificially incorporates several alternative forms of embodiment of the device of FIG. 3;

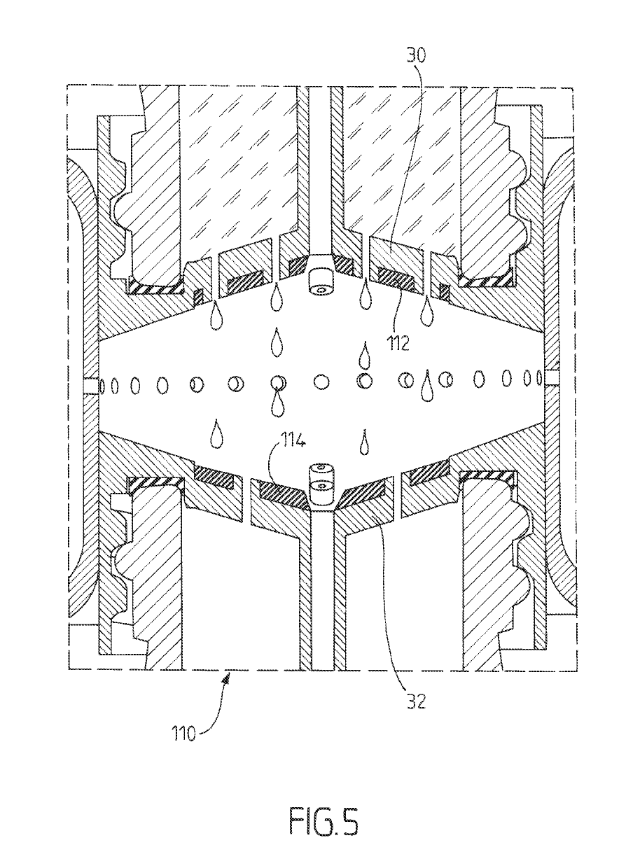

FIG. 5 is a schematic view in axial section of a fragrance diffusion device according to an additional alternative form of embodiment of the device of FIG. 1; and

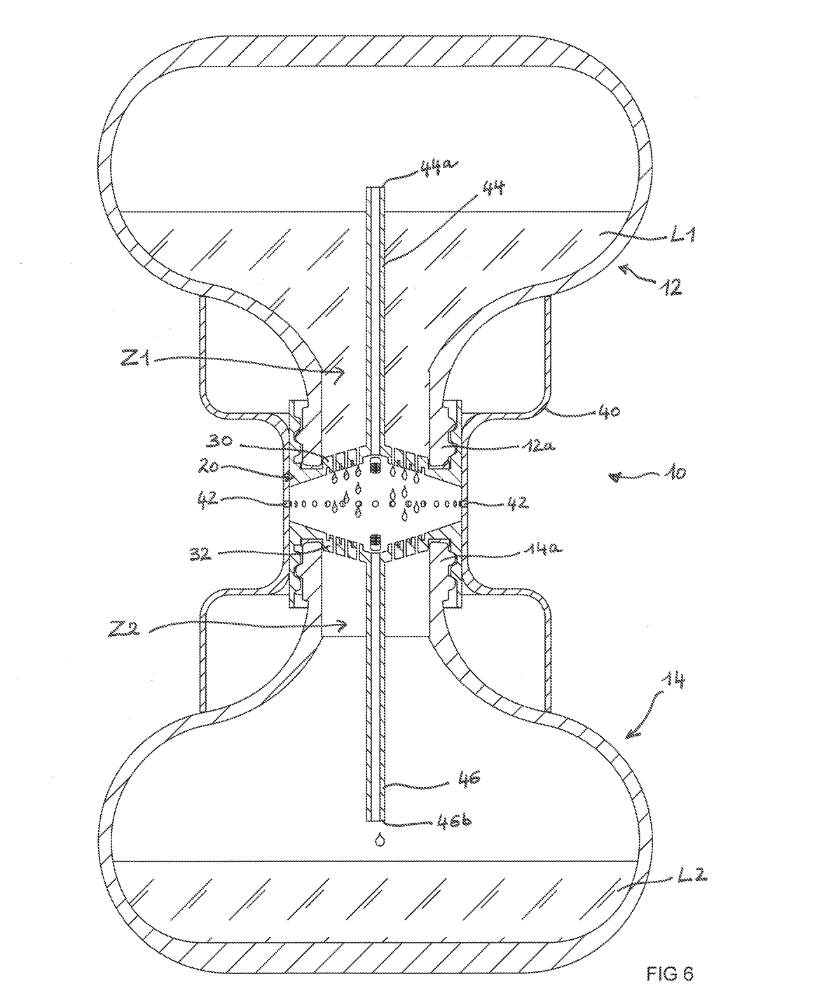

FIG. 6 is a schematic overall view of a fragrance diffusion system according to one embodiment of the invention.

As depicted partially in FIG. 1 and denoted by the general reference 10, a fragrance diffusion system according to one embodiment of the invention comprises a fragrance diffusion device 20 and two bottles 12, 14 mounted head to tail, only the respective necks 12a, 14a of which have been depicted. The system 10 is depicted in its entirety in FIG. 6 with the configuration of FIG. 2. However, the following description of FIGS. 1, 2 and 6 is not dependent on any particular configuration such as that of FIG. 2.

An upper first bottle 12 containing the fragrance (e.g. an ambient scent) that is to be diffused is mounted in an inverted position on top of the device 20 with its neck 12a oriented downwards.

The device 20 comprises first fixing means 22 which are intended to fix the neck 12a to said device. These fixing means are, for example, produced in the form of an internal screw thread 22a for screwing onto the external screw thread 12b on the external surface of the neck 12a.

A lower second bottle 14 intended to collect undiffused fragrance coming from the upper bottle 12 is mounted under the device 20 with its neck 14a oriented upwards.

The diffusion device 20 comprises second fixing means 24 which are intended to fix the neck 14a to said device. These fixing means are, for example, produced in the form of an internal screw thread 24a for screwing onto the external screw thread 14b on the external surface of the neck 14a. Other fixing means such as snap-fastening means may be envisaged.

The system 10 and its device 20 as well as the two bottles are arranged vertically in FIG. 1 so that the fragrance contained in the upper bottle 12 can flow under gravity.

The device 20 comprises two components, namely an upper component 26 and a lower component 28 positioned facing one another and spaced apart from one another along a longitudinal axis L which in this instance is oriented vertically.

The two components 26, 28 each comprise a wall 30, 32 arranged the one facing the other and which between them, along the longitudinal axis L, define a central cavity C.

The walls 30, 32 extend transversely from their central region towards their periphery where a wall 36, 38 extends laterally. The two axially extending walls 36, 38 of the components 26, 28 extend in opposite directions and away from the cavity C. The fixing means 22, 24 are formed on the interior surface of these walls. It will be noted that the neck 12a, 14a of each bottle, once fixed to the device 20, presses against a respective seal J1, J2 formed in a recess of the external face 30a, 32a of the relevant transversely extending wall.

As depicted, each transverse wall 30, 32 has a concave overall shape with the concave side facing towards the cavity C. Each wall is more particularly in the shape of a funnel converging towards the central region.

Each component 26, 28 generally has axial symmetry of revolution, give or take a few embodiment details. Thus, the axially extending walls 36, 38 each have a substantially cylindrical shape, making them skirt like.

The device 20 also comprises at its periphery a peripheral wall 40 having a ring like overall shape surrounding the assembly formed of the two opposing components 26, 28 and of the separating central cavity C. This peripheral wall 40 forms a trim element and is fixed to each of the aforementioned assemblies for example by bonding, welding or some other suitable means.

The peripheral wall 40 is provided with at least one opening 42 for diffusing fragrance to outside the device. Said at least one opening 42 is made in that region of the wall that is situated around the cavity C so as to place this cavity in communication with the outside of the device. In practice, for better distribution of the diffusion of fragrance, several openings 42 are arranged around the periphery of the peripheral wall 40 as depicted in FIG. 1, in an equatorial region of the device.

Each of the transversally extending walls 30, 32 comprises a plurality of through-openings denoted O1, O2 respectively, each of which places a zone external to the device, respectively denoted Z1, Z2 and adjacent to the relevant wall 30, 32 in communication with the cavity C. As depicted in FIG. 1, each external zone Z1, Z2 is a zone internal to the neck of a bottle.

FIG. 2 depicts another possible way in which the openings O1 can be distributed in the wall 30. The openings are arranged in series each grouping several openings, the series being arranged in a radial layout. Other alternative configurations may be used (e.g.: a layout in concentric rings, etc.).

The openings arranged in the upper wall (in this instance the openings O1 in the wall 30) are dimensioned so that the drops G of fragrance coming from the zone Z1 can pass through them and drop into the cavity as illustrated in FIG. 1. The multitude of openings perforating the wall provides a given diffusion capability. This parameter is dependent on the envisaged applications.

It will be noted that each opening O1, O2 is extended on the side of the internal face 30b, 32b of the wall 30, 32 by a neck denoted c1, c2 respectively and which projects into the cavity in relation to the relevant wall. This configuration makes it possible to prevent the openings from becoming blocked when the drops of fragrance fall onto the wall 32 which acts as a collector (or onto the wall 30 when it in its turn is at the bottom and is therefore acting as a collector).

The openings O1 and O2 in the two opposing walls 30, 32 do not face one another but are transversely offset in order to reduce the risk of blockage if drops fall onto the lower openings.

Each of the transversely extending walls 30, 32 also comprises a duct denoted 44, 46 respectively, formed in the central region of the wall (at the bottom of the concavity) and passing through the latter so as to place the relevant adjacent zone (Z1, Z2) in communication with the cavity C. More specifically, each duct 44, 46 extends longitudinally (in this instance vertically) in the relevant adjacent zone in the manner of a chimney. The upper duct 44 extends inside the upper bottle 12 over sufficient height that the open end 44a of the upper duct 44 is always above the level of the liquid fragrance L1 contained in the zone Z1 (FIG. 6). The same is true of the duct 46 when the device is inverted and it becomes an upper duct. In FIGS. 1 and 3 to 5, the open end of the duct 44 has not been extended as it has in FIG. 6 for the sake of simplification.

At its opposite end to the open free end each duct opens into the internal face 30b, 32b of the relevant wall without, however, projecting into the cavity. The openings O1 and O2 therefore respectively surround the ducts 44 and 46.

The upper duct 44 acts as an air intake duct which takes air in directly from outside the device via the openings 42 and not from the lower bottle. This arrangement is more effective because it is less sensitive to the variations in pressure from one bottle to the other. These pressure variations may for example be generated when the top bottle is exposed to the sun in a window and that at the bottom to shade, or vice versa, or alternatively when the bottom bottle is placed on a radiator whereas the top one is exposed to a cold draught. The changes in temperature influence the pressure through an effect of expanding the alcohol bases present in scents. In the device according to the invention, the intake air is at a stable pressure because it is coming in from outside. It will be noted that the duct is dimensioned (notably in terms of the diameter thereof) to allow fragrance to drip drop by drop into the cavity, this dripping being suited to the number of openings in the wall and to the diameter of same and is satisfactory in terms of the diffusion power (ability to diffuse scented molecules into the atmosphere). Specifically, the flow rate and therefore the rate of drop by drop dripping is notably dependent on the ability of the device to take in air.

The lower duct 46 collects the drops which have fallen onto the inclined wall 32 and have been guided under gravity as far as said duct. The duct 46 (penetrates the lower bottle 14) thus allows fragrance to flow into the zone Z2 and allows fragrance that has not been diffused to flow into the lower bottle.

When the fragrance diffusion system 10 is in operation, the liquid (fragrance) present in zone Z1 of the inverted upper bottle 12 passes through the openings O1 then forms drops G as it escapes from said openings. The drops drop under gravity inside the cavity over the entire height thereof and burst as they reach the lower wall 32 of the collector. Diffusion of fragrance thus occurs as the drops drop but mainly as the drops burst against the wall 32. Diffusion takes place radially or transversely through the air present inside the cavity C (diffusion zone) then through the openings 42 to leave the device 20.

The drop by drop dripping thus established in the cavity creates something of a suction on the liquid in the bottle 12 and therefore creates a depression in said bottle (pumping effect). This depression is compensated for by the external air that enters the bottle via the air intake duct 44.

Not all of the liquid that flows in the form of drops into the cavity is diffused therein. Specifically, a proportion of the drops reaching the lower wall 32 (arranged at a slope towards the inlet 46a of the flow duct 46) is collected on this wall without being diffused. These drops flow along the wall as far as the duct 46, enter it and flow along inside it and then into the lower bottle 14, in the form of drop by drop dripping. When the “non-diffused” liquid has passed into the lower bottle the diffusion phenomenon ceases. The capacity of the lower bottle is dimensioned so that the level of the liquid L2 transferred into the bottle 14 is situated below the free lower end 46b of the duct (FIG. 6).

When there is no longer any liquid in the upper bottle 12 and the lower bottle 14 is (at least partially) full, the system is inverted: the lower bottle 14 becomes the upper bottle and vice versa, and the mode of operation described hereinabove is repeated.

It will be noted that the flow rate of the liquid and therefore the speed of the drop by drop dripping may be dependent on:
the calibration of the holes O1,
the viscosity of the scent,
the calibration of the air intake 44.

The colleting walls 30 and 32 are inclined towards their central region (bottom of the concavity) so as to be able to collect the liquid in the central duct 46. This also allows the openings O2 present in the wall 32 (and the same applies to the openings O1 present in the wall 30 when the system is inverted) to be arranged at a level above the low point of the wall surrounding the duct 46 and around which the liquid could accumulate. Such an arrangement considerably reduces the risk of liquid collected by the wall 32 obstructing the openings O2 while the system is in operation.

According to an alternative form which has not been depicted, several air intake ducts 44 (and, respectively, flow ducts 46) may be arranged on the wall 30 (and, respectively, 32).

FIG. 3 depicts a fragrance diffusion system 60 according to a second embodiment of the invention. This system 60 comprises a fragrance diffusion device 70 which differs from the device 20 of FIG. 1 mainly through the presence of obstacles or bodies that divert the flow of liquid fragrance inside the cavity C, such as tubes.

Elements identical in relation to FIGS. 1 and 2 will keep the same references and will not be described again.

The concave upper 72 and lower 74 walls, which are inclined towards the inlet of each respective duct 44, 46, are also provided with openings O′1, O′2 passing through the relevant wall in the thickness thereof, so as to allow drops to form and to pass from the adjacent zone Z1, Z2 to the cavity.

Several tubes 76 extend longitudinally into the cavity, each between two openings O′1 and O′2 which are situated facing one another and connected by a tube. Each tube is, for example, inserted via its two opposite ends into the two aligned openings O′1 and O′2 respectively. Alternatively, the tubes may be fixed to the relevant wall without being inserted into the openings.

Each tube 76 is hollow and comprises a cylindrical wall 78 which is pierced with a plurality of lateral orifices 80 which place the cavity and the inside of the tube in communication.

More particularly, the tube 76 is divided into two elongate compartments or chambers 82, 84 arranged one above the other and separated from one another by a transverse partition 86, for example mid-way up the height of the tube. The first compartment 82 communicates with the opening O′1 and the second compartment 84 communicates with the opening O′2.

The lateral orifices 80 which are situated lowest down in the first compartment 82 are preferably arranged some distance away from the dividing partition 86 so as to leave between them a space which may fill with liquid (buffer volume).

In operation, the liquid flows through the openings O′1 and enters the first compartments 82 of each tube. The liquid accumulates under gravity in the abovementioned space until it reaches the lowermost orifices 80 and leaves the tubes thereby. If the volume of liquid is greater, the compartment 82 fills more until the upper orifices 80 are reached and until it leaves via these orifices. It will be noted that the cross section of the lateral orifices 80 and the internal cross section of the compartments 82, 84 is dimensioned to suit the intended objective. Thus, for example, if very small cross sections are selected for the lateral orifices 80, the liquid level will be able to rise in the larger cross-section compartment 82 and fill it to the top. In that case, it is the cross section of the lateral orifices 80 that defines the rate of flow of the scent into the cavity. The drops of liquid leaving the tubes flow along the external face thereof and, in the case of some of them, diffuse into the cavity during this flow. Other drops which have not diffused run along the external face of the tubes 76, reach the sloping lower wall 74 (collector), slip along this wall towards the central duct 46 and pass through it to drop into the lower bottle.

The tubes 76 and the lower wall 74 act as liquid collectors.

Moreover, the tubes 76 form chicanes for the flow of liquid coming from the openings O′1, thus slowing this flow and therefore the drop by drop dripping phenomenon. It then follows that the time spent by the drops passing (flowing) inside the cavity is increased, making it possible to increase the diffusion time and the diffusion capacity of the device. Diffusion time can be managed by a suitable choice of the number of tubes 76 and cross section thereof. The two-compartment arrangement described hereinabove is easy to clean in so far as the two compartments are accessible via their respective open ends.

According to possible alternative forms that have not been depicted:
the height of the compartments may differ from one compartment to the other, the dividing partition then being arranged differently;
the layout of the orifices may be different, for example in a staggered configuration;
the number and distribution of tubes in the cavity may differ, but still be suited to the number and arrangement of openings in the opposing walls of the device;
more than two compartments may be arranged one on top of another in order to increase the chicane effect on the flow.

It will be noted that other flow diverting bodies or obstacles such as means forming chicanes for diverting and therefore retarding the flow may alternatively be arranged in the cavity in relation, or not, with tubes such as dropping tubes 76.

FIG. 4 is a schematic view in axial section of a fragrance diffusion device 100 which artificially incorporates several different types of flow diverting bodies or obstacles (flow-retarding elements) which constitute alternative forms of the tubes of FIG. 3.

For the convenience of the explanation, the various types have been combined into the same central cavity C but a device 100 may very well comprise just one single type of flow-retarding element.

Components which are identical to those of FIG. 3 keep the same references.

In this alternative form, the function of the elements is to keep the liquid flowing from the upper bottle in the cavity C (diffusion chamber) for as long as possible by diverting its path along various simultaneous paths. The drops flow along these elements and remain on them even if the device is inverted (to limit runs).

These elongate elements are mounted via their upper and lower opposing ends in the corresponding walls 72 and 74.

These elongate elements illustrated in FIG. 4 are:
an element 102 of simple torpedo shape (smooth cylinder pointed at both ends),
a torpedo-shaped element 104 provided with studs 104a on the external longitudinal surface thereof,
a torpedo-shaped element 106 around which a spiral spring 106a is fitted,
an element 108 in the form of a helix.

FIG. 5 illustrates a device 110 according to an alternative form of embodiment of the device of FIG. 1. In the device 110, elements 112, 114 made of absorbent material (e.g. felt pellets) are formed on the walls 30, 32 (collectors), in order temporarily to hold the liquid (scent) there.

This material collects the liquid and thus remains moist. When the absorbent material 114 of the lower wall is saturated with liquid, the natural flow of liquid is directed toward the central duct 46.

Thus, when the device is inverted in the course of use, the liquid is held back by the absorbent material and therefore does not flood the cavity (greatly limiting runs).

In addition, the liquid (scent) is kept for longer in the cavity by these elements made of absorbent material (the retarding effect of the elements) and this contributes to improving the olfactory performance of the device.

It will be noted that the system depicted in FIG. 6 also applies to the various embodiments and alternative forms of embodiment described with reference to FIGS. 3 to 5 except for the structure of the fragrance-diffusion device.

1. Fragrance diffusion device (20; 70), which comprises:
two opposing walls (30, 32; 72, 74) spaced apart from one another along a vertical longitudinal axis (L) so as to define between them a cavity (C) which extends transversally as far as a peripheral wall (40) provided with at least one opening (42) for diffusing fragrance to outside the device, said at least one opening placing the cavity in communication with the outside of the device,
several openings (O1, O2; O′1, O′2) formed in each of the two opposing walls and which pass through the relevant wall in such a way as to allow, in the case of a first (30) of the two walls which is situated above the second wall (32), the passage of the liquid fragrance from a first zone (Z1) into the cavity so that said fragrance can be diffused to outside the device via said at least one diffusion opening, the first zone being adjacent to said first wall, above the latter and separated from the cavity by this first wall and, in the case of the second (32) of the two walls, the passage of the liquid fragrance from the cavity to a second zone (Z2) which is adjacent to said second wall, underneath the latter and separated from the cavity by this second wall,
at least one duct (44, 46) which passes through each of the two opposing walls and extends into the zone adjacent to the relevant wall so as to place each zone adjacent to each wall in communication with the cavity, said at least one duct formed in the first wall (30) being an air intake duct (44) which directly takes in air from the outside of the device via said at least one opening (42) and the cavity, whereas said at least one duct (46) formed in the second wall (32) is a flow duct for the liquid fragrance coming from the cavity.

2. Device according to claim 1, wherein said at least one air intake duct (44) extends in the first zone (Z1) over a height suited to the open end (44a) of said at least one air intake duct being at a position above the level of liquid fragrance contained in the first zone (Z1).

3. Device according to claim 1, wherein at least certain openings formed in the two opposing walls are each extended by a neck which projects into the cavity with respect to the relevant wall.

4. Device according to claim 1, wherein the openings (O1, O2; O′1, O′2) of one of the two opposing walls are geometrically offset in relation to the openings of the other wall.

5. Device according to claim 1, further comprising several liquid-fragrance flow-retarding elements (76; 102, 104, 106, 108) each comprising a body of elongate overall shape, the bodies extending into the cavity so as each to connect two respective openings of the two opposing walls, thus allowing the liquid fragrance to be guided via each flow-retarding element from an opening in the first wall (30) to an opening in the second wall (32).

6. Device according to claim 5, wherein the bodies of the fragrance flow-retarding elements are formed of several tubes (76) which extend into the cavity in such a way that each tube connects together two respective openings of the two opposing walls, thus allowing the liquid fragrance to be guided via each tube from an opening in the first wall to an opening in the second wall.

7. Device according to claim 1, wherein each of the two opposing walls (30, 32; 72, 74) has a concave overall shape with the concave side facing towards the cavity and comprises a central region.

8. Device according to claim 1, wherein said at least one duct (44, 46) is situated in the central region.

9. Device according to claim 5, wherein the at least one duct (44, 46) is situated in the central region, and the liquid-fragrance flow-retarding elements (76; 102, 104, 106, 108) are arranged around said at least one duct.

10. Device according to claim 1, further comprising one or more elements (112, 114) made of absorbent material, which are formed on each of the two opposing walls (30, 32).

11. Fragrance diffusion system (10; 60), which comprises:
a diffusion device (20; 70) according to claim 1,
an upper first bottle (12) having a first neck (12a) the opening of which is positioned facing the first (30; 72) of the two opposing walls referred to as the upper wall; a lower second bottle (14) having a second neck (14a) the opening of which is positioned facing the second wall referred to as the lower wall (32; 74), the upper first bottle comprising the liquid fragrance which, by flowing drop by drop through the openings (O1, O2; O′1, O′2) on the one hand causes the fragrance to diffuse into the cavity (C) and to outside of the device via the said at least one opening (42), and on the other hand causes dripping drop by drop into the lower second bottle.

12. Method for diffusing a fragrance in a fragrance diffusion device (20; 70), wherein the device comprises:
two opposing walls (30, 32; 72, 74) spaced apart from one another along a vertical longitudinal axis (L) so as to define between them a cavity (C) which extends transversally as far as a peripheral wall (40) provided with at least one opening (42) for diffusing fragrance to outside the device, said at least one opening placing the cavity in communication with the outside of the device,
several openings (O1, O2; O′1, O′2) formed in each of the two opposing walls and which pass through the relevant wall in such a way as to allow, in the case of a first (30) of the two walls which is situated above the second wall (32), the passage of the liquid fragrance from a first zone (Z1) into the cavity so that said fragrance can be diffused to outside the device via said at least one diffusion opening, the first zone being adjacent to said first wall, above the latter and separated from the cavity by this first wall and, in the case of the second (32) of the two walls, the passage of the liquid fragrance from the cavity to a second zone (Z2) which is adjacent to said second wall, underneath the latter and separated from the cavity by this second wall,
at least one duct (44, 46) which passes through each of the two opposing walls and extends into the zone adjacent to the relevant wall so as to place each zone adjacent to each wall in communication with the cavity, said at least one duct formed in the first wall (30) being an air intake duct (44) whereas said at least one duct (46) formed in the second wall (32) is a flow duct for the fragrance coming from the cavity, according to the method, the liquid fragrance contained in the first zone (Z1) flows through the openings (O1; O′1) in the first wall (30) so that, on the one hand, the fragrance diffuses into the cavity (C) and to outside the device via said at least one opening (42) and, on the other hand, the remaining liquid fragrance that has not diffused flows into the second zone (Z2) via said at least one duct (46), the flow of liquid fragrance through the openings in the first wall (30) being permitted as a result of the intake of external air via said at least one opening (42), the cavity (C) and said at least one air intake duct (44).

13. Method according to claim 12, wherein the flow of liquid fragrance from the first zone (Z1) through the openings (O1; O′1) in the first wall (30) is diverted, as it passes into the cavity, from a direct path in order to reach the second wall (32) so as to increase the time for which said flow is present in said cavity.

14. Method according to claim 13, wherein the flow of liquid fragrance is diverted by encountering a number of obstacles (76; 102; 104; 106; 108) present in the cavity (C).

15. Method according to claim 12, characterized in that the diffusion of fragrance takes place in a fragrance diffusion system (10; 60) comprising:
a fragrance diffusion device (20; 70),
an upper first bottle (12) having a first neck (12a) the opening of which is positioned facing the first (30; 72) of the two opposing walls referred to as the upper wall; a lower second bottle (14) having a second neck (14a) the opening of which is positioned facing the second wall referred to as the lower wall (32; 74), the upper first bottle comprising the fragrance.

16. Device according to claim 2, wherein at least certain openings formed in the two opposing walls are each extended by a neck which projects into the cavity with respect to the relevant wall.

17. Device according to claim 2, wherein the openings (O1, O2; O′1, O′2) of one of the two opposing walls are geometrically offset in relation to the openings of the other wall.

18. Device according to claim 2, further comprising several liquid-fragrance flow-retarding elements (76; 102, 104, 106, 108) each comprising a body of elongate overall shape, the bodies extending into the cavity so as each to connect two respective openings of the two opposing walls, thus allowing the liquid fragrance to be guided via each flow-retarding element from an opening in the first wall (30) to an opening in the second wall (32).
